(19) United States
(12) Patent Application Publication
(10) Pub. No.: US 2023/0046072 A1
(43) Pub. Date: Feb. 16, 2023

(54) METHOD FOR EVALUATING BIOLOGICAL EFFECT OF FAR INFRARED

(71) Applicant: National Taiwan University of Science and Technology, Taipei (TW)

(72) Inventors: San-Liang Lee, Taipei (TW); Yung-Ho Hsu, Taipei City (TW); Cheng-Hsien Chen, Taipei City (TW)

(73) Assignee: National Taiwan University of Science and Technology, Taipei (TW)

(21) Appl. No.: 17/742,411

(22) Filed: May 12, 2022

(30) Foreign Application Priority Data
Aug. 10, 2021 (TW) 110129527

(51) Int. Cl. A61B 5/145

(52) U.S. Cl. CPC A61B 5/14532; A61B 2503/40; A61B 2503/42

(57) ABSTRACT

A method for evaluating a biological effect of far infrared (FIR) includes the following. An FIR radiation source is provided to emit FIR. An FIR biological effect index (FBI) of the FIR is measured. A ratio of a blood glucose level change of an experimental group irradiated with the FIR to a blood glucose level change of a control group not irradiated with FIR is defined as the FBI. When the FBI is greater than 1, it is evaluated that the FIR causes a biological effect on an organism.

### CROSS-REFERENCE TO RELATED APPLICATION

This application claims the priority benefit of Taiwanese application no. 110129527, filed on Aug. 10, 2021. The entirety of the above-mentioned patent application is hereby incorporated by reference herein and made a part of this specification.

### BACKGROUND

### Technical Field

The disclosure relates to an evaluation method. In particular, the disclosure relates to a method for evaluating a biological effect of far infrared.

### Description of Related Art

Generally speaking, far infrared (FIR, at a wavelength of 3 micrometers (μm) to 1000 μm) produces many beneficial biological effects in terms of health and cell physiology, and these biological effects of the FIR are not related to thermal effects. Although there are many medical and healthcare devices that emit FIR on the market, appropriate ways to measure the biological effects of the FIR emitted by the devices are insufficient, consequently hindering verification on the effects of the devices, or even limiting applications of FIR in the respect of biomedicine.

### SUMMARY

The disclosure provides a method for evaluating a biological effect of far infrared, which may serve as a standardized method for measuring a far infrared biological effect index of a far infrared radiation source, and for evaluating a degree of a biological effect caused by far infrared thereof.

A method for evaluating a biological effect of far infrared of the disclosure includes the following. A far infrared radiation source is provided to emit far infrared. A far infrared biological effect index of the far infrared is measured. When the far infrared biological effect index is greater than 1, it is evaluated that the far infrared causes a biological effect on an organism.

In an embodiment of the disclosure, the method for measuring the far infrared biological effect index includes the following. Laboratory mice are provided. The laboratory mice are randomly divided into an experimental group and a control group. A glucose solution is fed to the experimental group and to the control group. A blood glucose level of the experimental group and a blood glucose level of the control group are measured at a first time point after the feeding. The experimental group is irradiated with the far infrared at the first time point, and the irradiating is continued until a second time point. The blood glucose level of the experimental group and the blood glucose level of the control group are measured at the second time point. A blood glucose level change from the first time point to the second time point of the experimental group is taken as a first difference. A blood glucose level change from the first time point to the second time point of the control group is taken as a second difference. A ratio of the first difference to the second difference is calculated, and the ratio is defined as the far infrared biological effect index.

In an embodiment of the disclosure, weeks of age of the laboratory mice are 10 weeks to 16 weeks.

In an embodiment of the disclosure, the first time point is a 15th minute after feeding the glucose solution.

In an embodiment of the disclosure, a time difference between the first time point and the second time point is 30 minutes.

In some embodiments of the disclosure, the biological effect of the far infrared is a non-thermal biological effect.

In some embodiments of the disclosure, the far infrared biological effect index is positively correlated with the biological effect.

In an embodiment of the disclosure, the biological effect includes reducing a blood glucose level, increasing a blood flow volume, and reducing a cardiovascular disease risk factor.

In an embodiment of the disclosure, when the far infrared has a wavelength between 8 micrometers and 10 micrometers and a radiation intensity between 0.087 milliwatt/square centimeter and 0.13 milliwatt/square centimeter, the far infrared causes the biological effect on the organism.

Based on the foregoing, in the method for evaluating a biological effect of far infrared according to the embodiments of the disclosure, since the ratio of the blood glucose level change of the experimental group to the blood glucose level change of the control group may be defined as the far infrared biological effect index, the measured and quantified far infrared biological effect index may be utilized to evaluate the degree of the biological effect caused by far infrared. The evaluation method may serve as a standardized method for measuring a far infrared biological effect index of a far infrared radiation source on the market, and for evaluating a degree of a biological effect caused by far infrared thereof.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 is a schematic flowchart of a method for evaluating a biological effect of far infrared according to an embodiment of the disclosure.

FIG. 2 is a schematic flowchart of a method for measuring a far infrared biological effect index according to an embodiment of the disclosure.

FIG. 3 shows a measured influence of far infrared on a blood glucose level of a mouse.

FIG. 4 shows relationships between weeks of age of a mouse and a far infrared biological effect index.

FIG. 5 shows relationships between an intensity of far infrared and a far infrared biological effect index.

FIG. 6 shows a measured influence of far infrared on an abdominal blood flow volume of a cisplatin-treated mouse.

FIG. 7 shows a measured influence of far infrared on a cardiovascular disease risk factor of a cisplatin-treated mouse.

### DESCRIPTION OF THE EMBODIMENTS

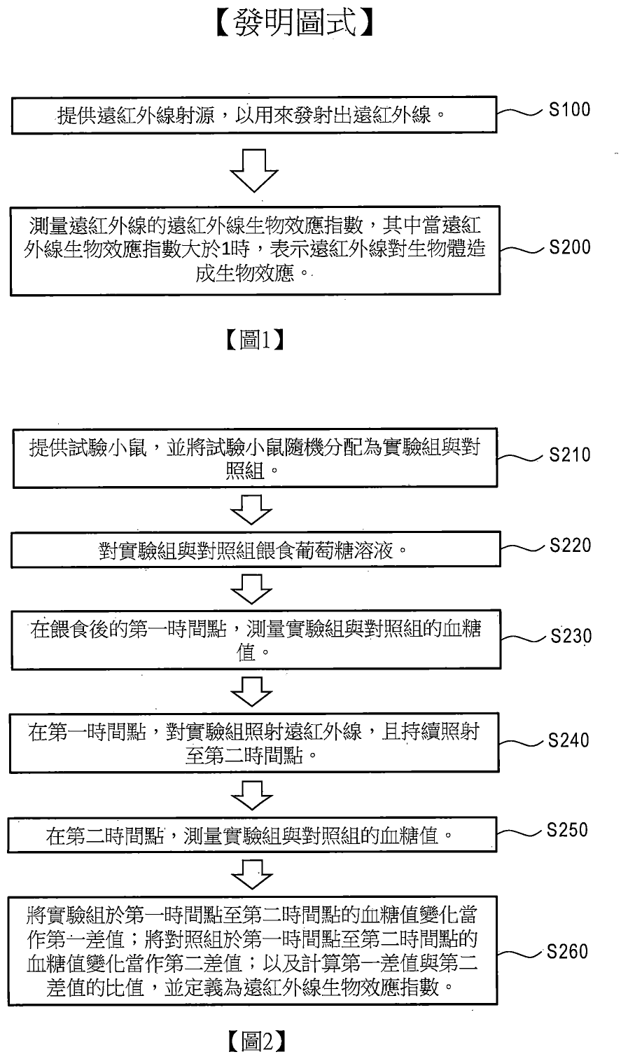

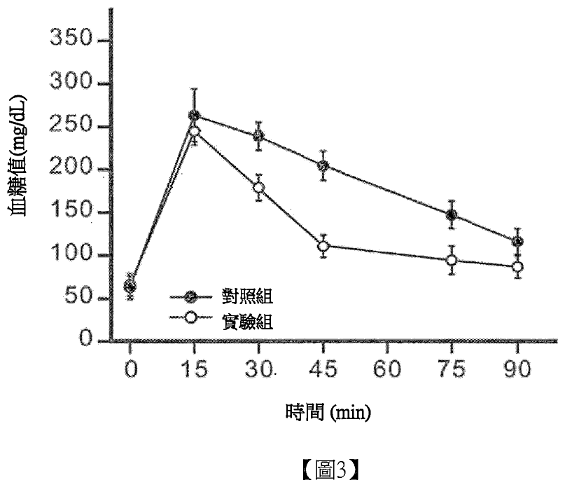

FIG. 1 is a schematic flowchart of a method for evaluating a biological effect of far infrared according to an embodiment of the disclosure. FIG. 2 is a schematic flowchart of a method for measuring a far infrared biological effect index according to an embodiment of the disclosure.

Generally speaking, far infrared in only a specific wavelength range resonates with molecules of an organism, affects physiological phenomena of the organism, and thus causes a biological effect on the organism. However, since far infrared emitted by a far infrared radiation source may have different wavelengths due to different compositions of the emitter thereof, even if far infrared is emitted at the same intensity, a degree of the biological effect caused by far infrared at different wavelengths among different far infrared radiation sources may not be the same. Therefore, this embodiment provides a method for evaluating a biological effect of far infrared that may be standardized. The biological effect of far infrared may include, for example but is not limited to, reducing a blood glucose level, increasing a blood flow volume, and reducing a cardiovascular disease risk factor.

In this embodiment, the method for evaluating a biological effect of far infrared may include the following. First, referring to FIG. 1, step S100 is performed to provide a far infrared radiation source to emit far infrared. The far infrared radiation source may be an emitter having far infrared radiation properties. For example, the far infrared radiation source may be, for example but not limited to, a far infrared therapeutic instrument, a far infrared protective gear, or a far infrared ceramic material.

Next, step S200 is performed to measure an FIR biological effect index (FBI) of the far infrared. Specifically, as shown in FIG. 2, in this embodiment, the method for measuring the FBI may include the following. First, step S210 is performed to provide laboratory mice. The laboratory mice are randomly divided into an experimental group and a control group. In this embodiment, the laboratory mice may be healthy mice, and weeks of age of the laboratory mice may be 8 weeks of age to 16 weeks of age, but the disclosure is not limited thereto. In some embodiments, the preferable weeks of age of the laboratory mice may be 10 weeks of age to 16 weeks of age.

Next, step S220 is performed to feed a glucose solution to the experimental group and to the control group. In this embodiment, the experimental group and the control group are fasted for approximately 12 hours before the glucose solution is fed, but not limited thereto. Here, the feeding volume of the glucose solution is 2 g/kg, but not limited thereto.

Next, step S230 is performed to measure a blood glucose level of the experimental group and a blood glucose level of the control group at a first time point after the feeding. In this embodiment, the first time point may be, for example but not limited to, the 15th minute after feeding the glucose solution. In addition, the method for measuring the blood glucose level may include, for example but is not limited to, first collecting a blood sample from the tail tip of the laboratory mouse, and then measuring the blood glucose level in the blood sample utilizing a glucometer.

Next, step S240 is performed to irradiate the experimental group with the far infrared at the first time point. The irradiating is continued until a second time point. In this embodiment, only the experimental group, but not the control group, is irradiated with far infrared. In addition, the second time point may be, for example but not limited to, the 45th minute after feeding the glucose solution. Therefore, in this embodiment, the experimental group may be continuously irradiated with the far infrared for 30 minutes. In other words, the time difference between the first time point and the second time point may be approximately 30 minutes, but is not limited thereto.

Next, step S250 is performed to measure the blood glucose level of the experimental group and the blood glucose level of the control group at the second time point. Here, the blood glucose level of the experimental group and the blood glucose level of the control group are measured by the method for measuring the blood glucose levels of step S230 above.

Next, step S260 is performed to take a blood glucose level change from the first time point to the second time point of the experimental group as a first difference, and take a blood glucose level change from the first time point to the second time point of the control group as a second difference. After that, a ratio of the first difference to the second difference is calculated, and the ratio is defined as the FBI. Therefore, in this embodiment, the formula for calculating the FBI is: the FBI=the blood glucose level change from the first time point to the second time point of the experimental group (i.e., the first difference)/the blood glucose level change from the first time point to the second time point of the control group (i.e., the second difference). So far, the FBI may have already be measured and calculated utilizing steps S210 to S260 above.

Then, with reference to FIG. 1, a biological effect of the far infrared is evaluated according to the measured FBI. In this embodiment, when the FBI is greater than 1, it may be evaluated that the far infrared emitted by the far infrared radiation source causes a biological effect on an organism. The relationship between the FBI and the biological effect may be a positive correlation. In other words, as the FBI increases, the biological effect also increases. In addition, in this embodiment, the biological effect of the far infrared is a non-thermal biological effect.

The technical means adopted by the disclosure to achieve the purpose in conjunction with the drawings and the embodiments will be described in the following. However, the following embodiments and accompanying drawings only serve for aiding the description, instead of limiting the disclosure.

### EMBODIMENTS

### Experimental Example 1: Evaluating the Biological Effect of Far Infrared and Measuring the FBI

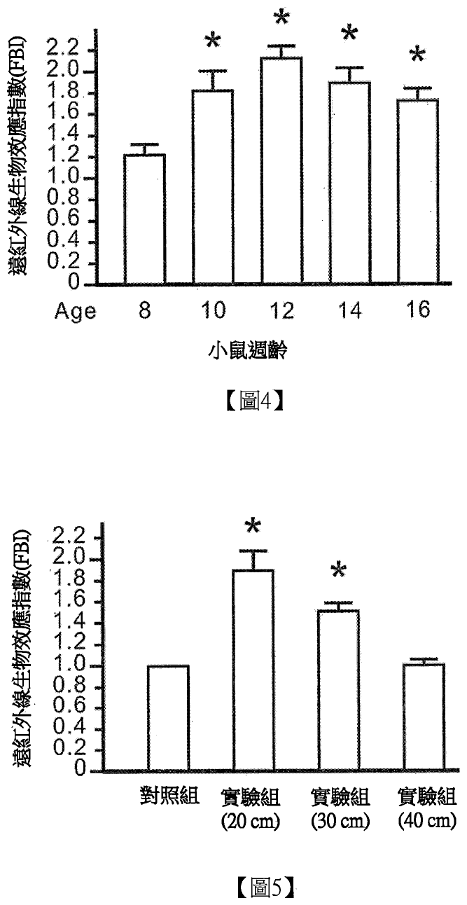

In this Experimental Example, male 129S1 mice at 12 weeks of age were first divided into an experimental group and an control group, with 5 mice in each group. Next, after 12 hours of fasting, a small amount of blood samples were collected from the tail tips of the mice in the experimental group and the control group, and blood glucose levels of the mice on an empty stomach (i.e., blood glucose levels at the 0th minute) were measured utilizing a glucometer. Then, 2 g/kg of a glucose solution (D-(+)-glucose dissolved into water) was fed from mouth (tube feeding) to the experimental group and the control group, and the blood glucose levels of the experimental group and the control group were measured at the 15th, 30th, 45th, 75th, and 90th minute after the feeding. In addition, at the 15th minute after the feeding, far infrared (at a wavelength of 8 μm to 10 μm) emitted by a far infrared radiation source (instrument: WS TY101 FIR emitter) was also used to irradiate the mice in the experimental group. At an irradiation distance of 20 centimeters (cm), the mice in the experimental group were exposed to far infrared with a radiation intensity of 0.13 milliwatt/square centimeter (mW/cm²). Lastly, the relationship between the measured blood glucose levels and the time points was recorded in Table 1 and illustrated into FIG. 3.

TABLE 1

| | 0 | 15 | 30 | 45 | 75 | 90 |
|---|---|---|---|---|---|---|
| Control | 63.1 ± 8.5 | 263.2 ± 33.4 | 238.1 ± 13.6 | 203.5 ± 19.2 | 147.4 ± 10.8 | 109.6 ± 11.3 |
| group (mg/dL) | | | | | | |
| Experimental | 65.4 ± 8.9 | 242.5 ± 12.3 | 174.9 ± 13.7 | 110.3 ± 12.8 | 90.9 ± 17.7 | 82.0 ± 10.1 |
| group (mg/dL) | | | | | | |

As can be seen from the results of Table 1 and FIG. 3, the experimental group irradiated with far infrared has a blood glucose level at the 30th, 45th, and 75th minute significantly lower than that of the control group not irradiated with far infrared. At the 30th minute, the difference between the blood glucose level of the control group and the blood glucose level of the experimental group is approximately 63 milligrams/deciliter (mg/dL). At the 45th minute, the difference between the blood glucose level of the control group and the blood glucose level of the experimental group is approximately 93 mg/dL. At the 75th minute, the difference between the blood glucose level of the control group and the blood glucose level of the experimental group is approximately 56 mg/dL. According to the above, the difference between the blood glucose level of the control group and the blood glucose level of the experimental group at the 45th minute is more significant than the difference between the blood glucose level of the control group and the blood glucose level of the experimental group at the 30th minute (or at the 75th minute). Therefore, in this Experimental Example, 30 minutes of far infrared irradiation (i.e., the 45th minute minus the 15th minute) was taken as the standard test time for measuring the FBI.

Next, a blood glucose level change from the 15th minute to the 45th minute of the experimental group was calculated to be approximately 132 mg/dL (i.e., the first difference), and a blood glucose level change from the 15th minute to the 45th minute of the mice in the control group was calculated to be approximately 60 mg/dL (i.e., the second difference). Then, by dividing the first difference by the second difference, the FBI can be obtained to be approximately 2.2 (132/60=2.2).

In this Experimental Example, since the measured and calculated FBI (approximately 2.2) may be greater than 1, it may be evaluated that far infrared has a biological effect of reducing a blood glucose level for the experimental group, as shown in FIG. 3. In other words, in this Experimental Example, a degree of the biological effect caused by far infrared may be evaluated utilizing the measured and quantified FBI.

Also, in this Experimental Example, since the mice need not be sacrificed after the experiment and may be used many times, and the use of expensive equipment is not required during the experiment, the method for evaluating and the method for measuring the FBI of this Experimental Example have low operation costs. In this Experimental Example, since the tube feeding technique and the blood collection technique in the experiment are easy, and the whole operation process only takes approximately 2 hours (in addition to the 12 hours of fasting in advance), the method for evaluating and the method for measuring the FBI of this Experimental Example have easy operation and low time costs.

In addition, in this Experimental Example, the method for evaluating and the method for measuring the FBI may serve as a standardized method for measuring an FBI of a far infrared radiation source on the market, and for evaluating a degree of a biological effect caused by far infrared thereof.

### Experimental Example 2: Measuring an Influence of Weeks of Age of a Mouse on the FBI

In this Experimental Example, in a way similar to Experimental Example 1, male 129S1 mice at 8 weeks of age, 10 weeks of age, 12 weeks of age, 14 weeks of age, and 16 weeks of age (5 mice at each of the weeks of ages) were exposed to far infrared with a radiation intensity of 0.13 mW/cm² for 30 minutes, to measure the influence of weeks of age of a mouse on the FBI. Lastly, the relationship between the measured and calculated FBIs and the weeks of age was illustrated into FIG. 4.

With reference to FIG. 4, in this Experimental Example, after comparison and statistical analysis of the FBI for mice at 10, 12, 14, or 16 weeks of age and the FBI for mice at 8 weeks of age, if the results of statistical analysis show a significant difference and the p-value is less than 0.01, it is indicated by an asterisk * in the drawing.

As can be seen from the results of FIG. 4, the FBI for the mice at 8 weeks of age is 1.2±0.1, the FBI for the mice at 10 weeks of age is 1.8±0.2, the FBI for the mice at 12 weeks of age is 2.1±0.1, the FBI for the mice at 14 weeks of age is 1.940.1, and the FBI for the mice at 16 weeks of age was 1.7±0.1. The mice at 10, 12, 14, and 16 weeks of age each have a significantly higher FBI that that of the mice at 8 weeks of age.

### Experimental Example 3: Measuring an Influence of Intensity of Far Infrared on the FBI

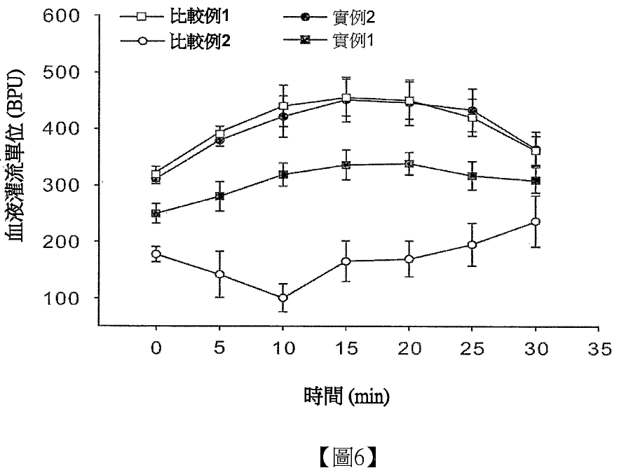

In this Experimental Example, in a way similar to Experimental Example 1, male 129S1 mice at 10 weeks of age were divided into 3 experimental groups and 1 control group, with 5 mice in each group. Next, the three experimental groups were irradiated with far infrared for 30 minutes at different irradiation distances (irradiation distances of 20 cm, 30 cm, or 40 cm), to measure the influence of the intensity of far infrared on the FBI. At the irradiation distance of 20 cm, the mice were exposed to far infrared with a radiation intensity of 0.13 mW/cm². At the irradiation distance of 30 cm, the mice were exposed to far infrared with a radiation intensity of 0.087 mW/cm². At the irradiation distance of 40 cm, the mice were exposed to far infrared with a radiation intensity of 0.033 mW/cm². Lastly, the relationship between the measured and calculated FBIs and the irradiation distances was illustrated into FIG. 5.

As can be seen from the results of FIG. 5, the FBI for the control group should be 1, the FBI at the irradiation distance of 20 cm is 1.9±0.2, the FBI at the irradiation distance of 30 cm is 1.5±0.1, and the FBI for the mice at the irradiation distance of 40 cm is 140.05. In other words, in this Experimental Example, with the use of WS TY101 FIR emitter that emits far infrared at a wavelength of 8 μm to 10 μm, the condition for reaching an FBI of 1.9 (or 1.5) is to irradiate mice at a distance of 20 cm (or 30 cm) to expose the mice to far infrared with a radiation intensity of 0.13 mW/cm² (or 0.087 mW/cm²). However, it should be noted that differences in the material of the emitter and/or the operating temperature of the radiation source in other far infrared radiation sources (or devices that emit far infrared) may cause the wavelength distribution and/or intensity of the emitted far infrared to be different from the wavelength distribution and/or intensity of the WS TY101 FIR emitter. Therefore, the condition for reaching an FBI of 1.9 (or 1.5) above is not necessarily applicable to other far infrared radiation sources (or devices that emit far infrared).

In this Experimental Example, after comparison and statistical analysis of the FBIs at the irradiation distances of 20 cm, 30 cm, or 40 cm and the FBI for the control group, if the results of statistical analysis show a significant difference and the p-value is less than 0.01, it is indicated by an asterisk * in the drawing. As shown in FIG. 5, the FBI is significantly higher at the irradiation distances of 20 cm and 30 cm than that for the control group.

### Experimental Example 4: Measuring an Influence of Far Infrared on an Abdominal Blood Flow Volume of a Cisplatin-Treated Mouse

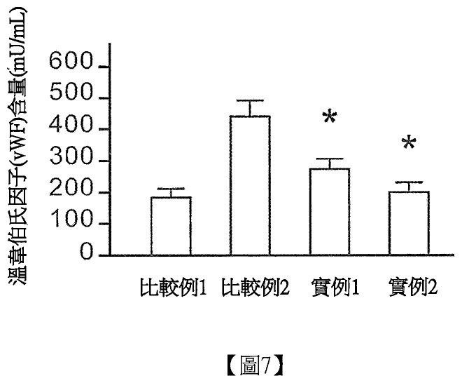

In this Experimental Example, C57BL/6J mice at 8 weeks of age were first divided into 2 experimental groups (Example 1, Example 2) and 2 control groups (Comparative Example 1, Comparative Example 2), with 5 mice in each group. Next, cisplatin (at a dose of 4 mg/kg) was injected from the tail veins into the mice of Example 1, Example 2, and Comparative Example 2 to decrease the blood flow volumes of Example 1, Example 2, and Comparative Example 2. Next, far infrared (including a wavelength of 8 μm to 10 μm) emitted by a far infrared radiation source (instrument: WS TY101 FIR emitter) was used to irradiate Example 1 and Example 2 for 30 minutes a day for 3 consecutive days. Light emitted by a tungsten filament lamp (at a wavelength of 0.3 μm to 2.5 μm) was used to irradiate Comparative Example 2 for 30 minutes a day for 3 consecutive days. Example 1 was irradiated under the condition that the FBI was 1.5 (i.e., the mice were irradiated with far infrared at a wavelength of 8 μm to 10 μm at an irradiation distance of 30 cm). Example 2 was irradiated under the condition that the FBI was 1.9 (i.e., the mice were irradiated with far infrared at a wavelength of 8 μm to 10 μm at an irradiation distance of 20 cm). Moreover, Comparative Example 1 was neither injected with cisplatin nor irradiated using a far infrared radiation source or a tungsten filament lamp. Next, after the irradiation of the 3rd day, Example 1, Example 2, Comparative Example 1, and Comparative Example 2 were anesthetized (with isoflurane) and covered with a heating blanket at 40° C. Then, abdominal blood flow volumes of the mice were detected with a laser Doppler detector during a period of 30 minutes. Lastly, the relationship between the measured blood flow volumes (represented by blood perfusion units (BPU)) and the time points was illustrated into FIG. 6.

As can be seen from the results of FIG. 6, in Comparative Example 2 irradiated by a tungsten filament lamp, since the mice injected with cisplatin had a decreased blood flow volume and even clogged small blood vessels due to damaged blood vessels, the blood flow volume could not be increased by heating, and was even decreased after 10 minutes during the blood flow volume analysis. The blood flow volumes of both Example 1 and Example 2 irradiated by a far infrared radiation source are significantly higher than that of Comparative Example 2 irradiated by a tungsten filament lamp. Therefore, it may indicate that although far infrared of a far infrared radiation source and light of a tungsten filament lamp both release heat, only far infrared causes an increase in the blood flow volume (a biological effect) to Example 1 and Example 2. Therefore, it follows that the biological effect caused by far infrared is a non-thermal biological effect. In this Experimental Example, since the blood flow volume of Example 2 is significantly higher than the blood flow volume of Example 1, it may indicate that as the FBI increases, the blood flow volume also increases. In other words, the FBI is positively correlated with an increase in the blood flow volume (biological effect). In this Experimental Example, since the blood flow volume of Example 2 is similar to the blood flow volume of Comparative Example 1, it may indicate that irradiating a mouse injected with cisplatin under the condition that the FBI is 1.8 causes the blood flow volume of the mouse to return to a normal value.

### Experimental Example 5: Measuring an Influence of Far Infrared on a Cardiovascular Disease Risk Factor of a Cisplatin-Treated Mouse

In this Experimental Example, a way similar to Experimental Example 4 was performed on Example 1, Example 2, Comparative Example 1, and Comparative Example 2 to increase the contents of von Willebrand factor (VWF) in the plasma of Example 1, Example 2, and Comparative Example 2 after injected with cisplatin. Next, after the irradiation of the 3rd day, Example 1, Example 2, Comparative Example 1, and Comparative Example 2 were anesthetized (with isoflurane), and blood samples were obtained from cardiac exsanguination. Next, the contents of VWF in the plasma were measured by enzyme-linked immunosorbent assay (ELISA). Lastly, the measured contents of VWF of Example 1, Example 2, Comparative Example 1, and Comparative Example 2 were illustrated into FIG. 7. It is known that the content of VWF is one of the cardiovascular disease risk factors.

With reference to FIG. 7, in this Experimental Example, after comparison and statistical analysis of the contents of VWF of Example 1 and Example 2 and the content of VWF of Comparative Example 2, if the results of statistical analysis show a significant difference and the p-value is less than 0.01, it is indicated by an asterisk * in the drawing.

As can be seen from the results of FIG. 7, since comparative Example 2 (injected with cisplatin and irradiated with a tungsten filament lamp) has a significantly increased content of VWF compared with Comparative Example 1 (neither injected with cisplatin nor irradiated with a far infrared radiation source or a tungsten lamp), it may indicate that irradiation by a tungsten lamp does not reduce the content of VWF increased by cisplatin injection. In this Experimental Example, since the contents of VWF in both Example 1 and Example 2 irradiated by a far infrared radiation source are significantly lower than that of Comparative Example 2 irradiated by a tungsten lamp, it may indicate that although far infrared of a far infrared radiation source and light of a tungsten filament lamp both release heat, only far infrared causes a reduction in the cardiovascular disease risk factor (a biological effect) to Example 1 and Example 2. Therefore, it follows that the biological effect of far infrared is a non-thermal biological effect. In this Experimental Example, since the content of VWF of Example 2 is significantly lower than that of Example 1, it may indicate that as the FBI increases, the content of VWF also decreases. In other words, the FBI may be positively correlated with a reduction in the cardiovascular disease risk factor (biological effect). In this Experimental Example, since the content of VWF of Example 2 is similar to the content of VWF of Comparative Example 1, it may indicate that irradiating a mouse injected with cisplatin under the condition that the FBI is 1.8 causes the content of VWF of the mouse to return to a normal value.

In summary of the foregoing, in the method for evaluating a biological effect of far infrared according to the embodiments of the disclosure, since the ratio of the blood glucose level change of the experimental group to the blood glucose level change of the control group may be defined as the far infrared biological effect index, the measured and quantified far infrared biological effect index may be utilized to evaluate the degree of the biological effect caused by far infrared. When the far infrared biological effect index is greater than 1, it may be evaluated that the far infrared causes a biological effect on an organism. In the embodiments of the disclosure, since the mice need not be sacrificed after the experiment and may be used many times, and the use of expensive equipment is not required during the experiment, the evaluation method in Experimental Examples of the disclosure has low operation costs. In the embodiments of the disclosure, since the tube feeding technique and the blood collection technique in the experiment are easy, and the whole operation process only takes approximately 2 hours (in addition to the 12 hours of fasting in advance), the evaluation method in Experimental Examples of the disclosure has easy operation and low time costs. In addition, in the embodiments of the disclosure, the evaluation method may serve as a standardized method for measuring a far infrared biological effect index of a far infrared radiation source on the market, and for evaluating a degree of a biological effect caused by far infrared thereof.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A method for evaluating a biological effect of far infrared, comprising:
providing a far infrared radiation source to emit far infrared; and
measuring a far infrared biological effect index of the far infrared, wherein the method for measuring the far infrared biological effect index comprises:
providing laboratory mice, wherein the laboratory mice are randomly divided into an experimental group and a control group;
feeding a glucose solution to the experimental group and to the control group;
measuring a blood glucose level of the experimental group and a blood glucose level of the control group at a first time point after the feeding;
irradiating the experimental group with the far infrared at the first time point, wherein the irradiating is continued until a second time point;
measuring the blood glucose level of the experimental group and the blood glucose level of the control group at the second time point;
taking a blood glucose level change from the first time point to the second time point of the experimental group as a first difference;
taking a blood glucose level change from the first time point to the second time point of the control group as a second difference; and
calculating a ratio of the first difference to the second difference, and defining the ratio as the far infrared biological effect index,
wherein when the far infrared biological effect index is greater than 1, it is evaluated that the far infrared causes a biological effect on an organism.

2. The method according to claim 1, wherein weeks of age of the laboratory mice are 10 weeks to 16 weeks.

3. The method according to claim 1, wherein the first time point is a 15th minute after feeding the glucose solution.

4. The method according to claim 1, wherein a time difference between the first time point and the second time point is 30 minutes.

5. The method according to claim 1, wherein the biological effect of the far infrared is a non-thermal biological effect.

6. The method according to claim 1, wherein the far infrared biological effect index is positively correlated with the biological effect.

7. The method according to claim 1, wherein the biological effect comprises reducing a blood glucose level, increasing a blood flow volume, and reducing a cardiovascular disease risk factor.

8. The method according to claim 1, wherein when the far infrared has a wavelength between 8 micrometers and 10 micrometers and a radiation intensity between 0.087 milliwatt/square centimeter and 0.13 milliwatt/square centimeter, the far infrared causes the biological effect on the organism.
